(19) United States
(12) Patent Application Publication
(10) Pub. No.: US 2022/0181021 A1
(43) Pub. Date: Jun. 9, 2022

(54) INFORMATION PROCESSING APPARATUS, NON-TRANSITORY COMPUTER READABLE MEDIUM, AND INFORMATION PROCESSING METHOD

(71) Applicant: TOYOTA JIDOSHA KABUSHIKI KAISHA, Aichi-ken (JP)

(72) Inventors: Toyokazu NAKASHIMA, Nagoya-shi (JP); Shunsuke SAGARA, Nisshin-shi (JP); Naoya OKA, Nagakute-shi (JP)

(21) Appl. No.: 17/643,181

(22) Filed: Dec. 8, 2021

(30) Foreign Application Priority Data
Dec. 9, 2020 (JP) 2020-204429

(51) Int. Cl. G16H 40/67; H04L 67/12; H04M 3/51

(52) U.S. Cl. CPC G16H 40/67; H04M 3/5116; H04L 67/12

(57) ABSTRACT

An information processing apparatus includes a controller configured to generate a message notifying a healthcare professional of information on a call concerning a patient, and transmit first information, to an emergency center, notifying that there is a healthcare professional who is going to the patient based on the message.

### CROSS-REFERENCE TO RELATED APPLICATION

This application claims priority to Japanese Patent Application No. 2020-204429, filed on Dec. 9, 2020, the entire contents of which are incorporated herein by reference.

### TECHNICAL FIELD

The present disclosure relates to an information processing apparatus, a program, and an information processing method.

### BACKGROUND

Rescue operations for patients requiring critical care need to be performed quickly.

Patent literature (PTL) 1 discloses a lifesaving assistance system that, upon detecting an abnormality in the physical condition of a user carrying a terminal apparatus, transmits lifesaving information, for saving the user's life, to a terminal apparatus of a third party located near the user.

### CITATION LIST

### Patent Literature

PTL 1: JP 2012-222443 A

### SUMMARY

PTL 1 does not disclose rescue operations being performed by collaboration between a third party located near the user and an ambulance in the lifesaving assistance system.

It would be helpful to perform rescue operations efficiently by collaboration between an ambulance and a healthcare professional other than the healthcare professional on board the ambulance.

An information processing apparatus according to the present disclosure includes a controller configured to: generate a message notifying a healthcare professional of information on a call concerning a patient; and transmit first information, to an emergency center, notifying that there is a healthcare professional who is going to the patient based on the message.

A program according to the present disclosure is configured to cause a computer to execute operations including: generating a message notifying a healthcare professional of information on a call concerning a patient; and transmitting first information, to an emergency center, notifying that there is a healthcare professional who is going to the patient based on the message.

An information processing method according to the present disclosure includes: generating a message notifying a healthcare professional of information on a call concerning a patient; and transmitting first information, to an emergency center, notifying that there is a healthcare professional who is going to the patient based on the message.

According to the present disclosure, rescue operations can be performed efficiently by collaboration between an ambulance and a healthcare professional other than the healthcare professional on board the ambulance.

### BRIEF DESCRIPTION OF THE DRAWINGS

In the accompanying drawings:

FIG. 1 is a diagram illustrating a configuration of an information processing system according to an embodiment of the present disclosure;

FIG. 2 is a block diagram illustrating a configuration of an information processing apparatus according to an embodiment of the present disclosure;

FIG. 3 is a block diagram illustrating a configuration of a vehicle according to an embodiment of the present disclosure;

FIG. 4 is a block diagram illustrating a configuration of a terminal apparatus according to the embodiment of the present disclosure; and

FIG. 5 is a flowchart illustrating operations of an information processing system according to an embodiment of the present disclosure.

### DETAILED DESCRIPTION

An embodiment of the present disclosure is described below with reference to the drawings.

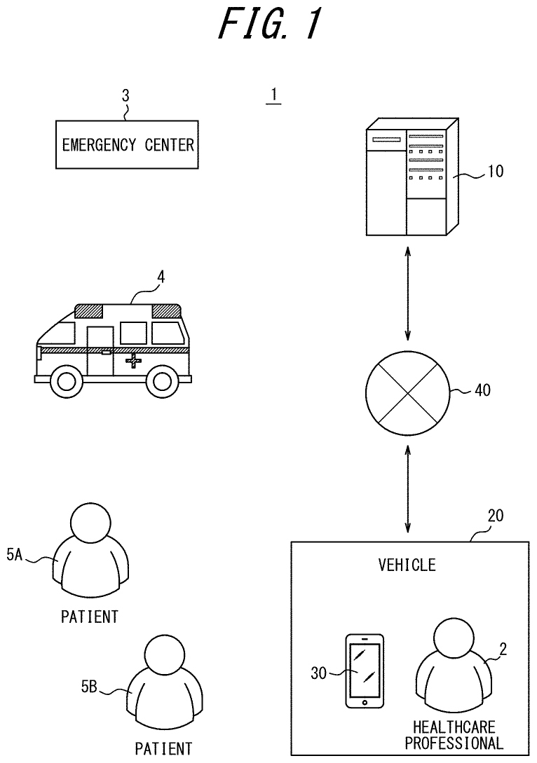

FIG. 1 is a diagram illustrating a configuration of an information processing system 1 according to an embodiment of the present disclosure. The configuration and outline of the information processing system 1 according to an embodiment of the present disclosure are described with reference to FIG. 1.

The information processing system 1 includes an information processing apparatus 10, a vehicle 20, and a terminal apparatus 30. The information processing apparatus 10, the vehicle 20, and the terminal apparatus 30 are communicably connected via a network 40. The network 40 may be a network including a mobile communication network, the Internet, and the like.

In FIG. 1, one each of the information processing apparatus 10, the vehicle 20, and the terminal apparatus 30 are illustrated, but the number of information processing apparatuses 10, vehicles 20, and terminal apparatuses 30 may be two or more of each.

As illustrated in FIG. 1, the vehicle 20 is a vehicle with a healthcare professional 2 on board. The terminal apparatus 30 is a terminal apparatus owned by the healthcare professional 2 who is on board the vehicle 20.

The healthcare professional 2 may be a doctor, nurse, paramedic, or the like.

An emergency center 3 is an organization that can handle emergency patients. An information communication device, such as a server, is installed in the emergency center 3. The information communication device installed in the emergency center 3 can communicate with the information processing apparatus 10 via the network 40.

An ambulance 4 goes to the patient 5 designated as the destination in response to a command from the emergency center 3. The paramedic on board the ambulance 4 provides first aid to the patient 5. The ambulance 4 transports the patient 5 to the hospital after the first aid.

Patients 5A, 5B are patients in a non-hospital setting, such as a home or the street. Two patients 5A, 5B are illustrated in FIG. 1, but the number of patients 5 is not limited to two. The number of patients 5 may be one or may be or three or more. In the description of the present embodiment, the patients 5A, 5B may simply be referred to as patient 5 when no particular distinction need be made.

The patient 5 calls the emergency center 3 to indicate that treatment is needed. The patient 5 need not be the one who calls the emergency center 3. For example, a third party who notices something wrong with the patient 5 may call the emergency center.

The information processing apparatus 10 is, for example, a dedicated computer configured to function as a server. The information processing apparatus 10 may be a general purpose personal computer (PC).

The information processing apparatus 10 can communicate with the vehicle 20 and the terminal apparatus 30 via the network 40. The information processing apparatus 10 can communicate with the information communication device installed in the emergency center 3 via the network 40. Upon acquiring information on the call concerning the patient 5 from the emergency center 3, the information processing apparatus 10 generates a message notifying the healthcare professional 2 of the information on the call concerning the patient 5 and transmits the message to the terminal apparatus 30 of the healthcare professional 2 or to the vehicle 20 that has the healthcare professional 2 on board. The “message notifying the healthcare professional 2 of the information on the call concerning the patient 5” is also referred to below simply as a “message”.

When a healthcare professional 2 who sees the message goes to the patient 5 based on the message, the information processing apparatus 10 transmits first information, which is information notifying that there is a healthcare professional 2 who is going to the patient 5 based on the message, to the information communication device installed in the emergency center 3. In subsequent explanation, “transmitting to the information communication device installed in the emergency center 3” may be described simply as “transmitting to the emergency center 3”.

The vehicle 20 can communicate with the information processing apparatus 10 and the terminal apparatus 30 via the network 40. The vehicle 20 may be any type of automobile, such as a gasoline-powered vehicle, a diesel-powered vehicle, an HEV (Hybrid Electric Vehicle), a PHEV (Plug-in Hybrid Electric Vehicle), a BEV (Battery Electric Vehicle), a FCEV (Fuel Cell Electric Vehicle), or the like. Driving of the vehicle 20 may be automated at any level. The automation level is, for example, any one of Level 1 to Level 5 according to the level classification defined by the Society of Automotive Engineers (SAE). The vehicle 20 may be a dedicated Mobility as a Service (MaaS) vehicle.

The terminal apparatus 30 can communicate with the information processing apparatus 10 and the vehicle 20 via the network 40. The terminal apparatus 30 may, for example, be a smartphone or a tablet.

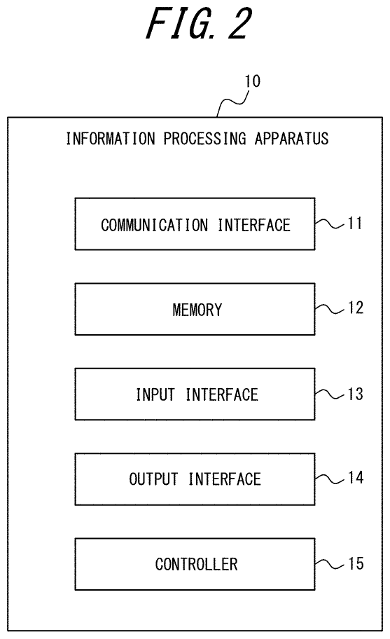

A configuration of the information processing apparatus 10 according to an embodiment of the present disclosure is described with reference to FIG. 2.

The information processing apparatus 10 includes a communication interface 11, a memory 12, an input interface 13, an output interface 14, and a controller 15.

The communication interface 11 includes a communication module connected to the network 40. For example, the communication interface 11 may include a communication module corresponding to a local area network (LAN). In an embodiment, the information processing apparatus 10 is connected to the network 40 via the communication interface 11. The communication interface 11 transmits and receives various information via the network 40. The communication interface 11 can communicate with the vehicle 20 and the terminal apparatus 30 via the network 40. The communication interface 11 is also capable of communicating with the information communication device installed in the emergency center 3 via the network 40.

The memory 12 is, for example, a semiconductor memory, a magnetic memory, an optical memory, or the like, but is not limited to these. The memory 12 may function as, for example, a main memory, an auxiliary memory, or a cache memory. The memory 12 stores any information used in operation of the information processing apparatus 10. For example, the memory 12 may store a system program, an application program, various types of information received by the communication interface 11, and the like. The information stored in the memory 12 may be updated with information received from the network 40 via the communication interface 11, for example. A portion of the memory 12 may be installed externally to the information processing apparatus 10. In this case, the externally installed portion of the memory 12 may be connected to the information processing apparatus 10 via any appropriate interface.

The input interface 13 includes one or more input interfaces for detecting user input and acquiring input information based on user operation. For example, the input interface 13 includes, but is not limited to, a physical key, a capacitive key, a touchscreen integrally provided with a display of the output interface 14, or a microphone that receives audio input.

The output interface 14 includes one or more output interfaces for outputting information to notify the user. For example, the output interface 14 includes, but is not limited to, a display for outputting information as images, a speaker for outputting information as audio, or the like.

The controller 15 includes at least one processor, at least one dedicated circuit, or a combination thereof. The processor is a general purpose processor, such as a central processing unit (CPU) or a graphics processing unit (GPU), or a dedicated processor specialized for particular processing. The dedicated circuit is, for example, a field-programmable gate array (FPGA) or an application specific integrated circuit (ASIC). The controller 15 executes processes related to operations of the information processing apparatus 10 while controlling components of the information processing apparatus 10.

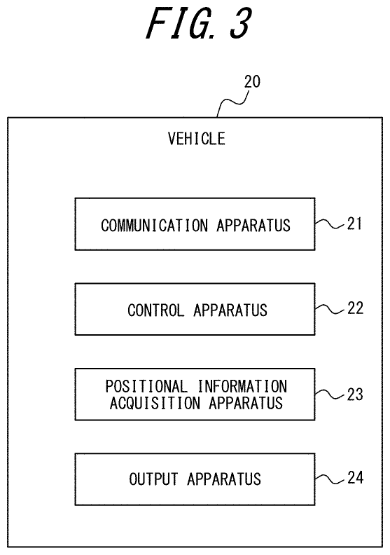

A configuration of the vehicle 20 according to an embodiment of the present disclosure is described with reference to FIG. 3.

The vehicle 20 includes a communication apparatus 21, a control apparatus 22, a positional information acquisition apparatus 23, and an output apparatus 24. The communication apparatus 21, the control apparatus 22, the positional information acquisition apparatus 23, and the output apparatus 24 are communicably connected via an in-vehicle network, such as Controller Area Network (CAN), or dedicated lines, for example.

The communication apparatus 21 includes a communication module that connects to the network 40. For example, the communication apparatus 21 may include a communication module compliant with mobile communication standards such as long term evolution (LTE) and the 4th generation (4G) and the 5th generation (5G) standards. In an embodiment, the vehicle 20 is connected to the network 40 via the communication apparatus 21. The communication apparatus 21 transmits and receives various information via the network 40. The communication apparatus 21 can communicate with the information processing apparatus 10 and the terminal apparatus 30 via the network 40.

The control apparatus 22 includes at least one processor, at least one dedicated circuit, or a combination thereof. The processor is a general purpose processor such as a CPU or a GPU, or a dedicated processor that is dedicated to specific processing. The dedicated circuit is, for example, an FPGA or an ASIC. The control apparatus 22 executes processing related to operations of the vehicle 20 while controlling each component of the vehicle 20.

The positional information acquisition apparatus 23 includes at least one receiver compliant with a satellite positioning system. For example, the positional information acquisition apparatus 23 may include a global positioning system (GPS) receiver. The positional information acquisition apparatus 23 acquires the measured value of the position of the vehicle 20 as the positional information. The positional information includes, for example, an address, a latitude, a longitude, an altitude, and the like.

The output apparatus 24 includes one or more output interfaces for outputting information to notify the user. For example, the output apparatus 24 includes, but is not limited to, a display for outputting information as video, a speaker for outputting information as audio, or the like.

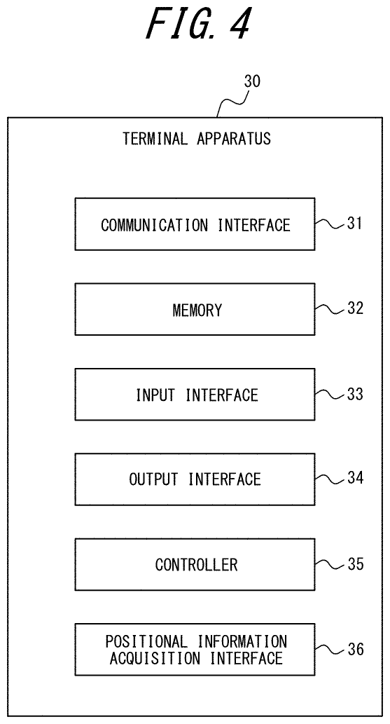

A configuration of the terminal apparatus 30 according to an embodiment of the present disclosure is described with reference to FIG. 4.

The terminal apparatus 30 includes a communication interface 31, a memory 32, an input interface 33, an output interface 34, a controller 35, and a positional information acquisition interface 36.

The communication interface 31 includes a communication module connected to the network 40. For example, the communication interface 31 may include a communication module compliant with mobile communication standards such as LTE, 4G and 5G. In an embodiment, the terminal apparatus 30 is connected to the network 40 via the communication interface 31. The communication interface 31 transmits and receives various information via the network 40. The communication interface 31 can communicate with the information processing apparatus 10 and the vehicle 20 via the network 40.

The memory 32 is, for example, a semiconductor memory, a magnetic memory, an optical memory, or the like, but is not limited to these. The memory 32 may function as, for example, a main memory, an auxiliary memory, or a cache memory. The memory 32 stores any information used for operations of the terminal apparatus 30. For example, the memory 32 may store a system program, an application program, various types of information received by the communication interface 31, and the like. The information stored in the memory 32 may be updated with information received from the network 40 via the communication interface 31, for example. A portion of the memory 32 may be installed externally to the terminal apparatus 30. In this case, the externally installed portion of the memory 32 may be connected to the terminal apparatus 30 via any appropriate interface.

The input interface 33 includes one or more input interfaces for detecting user input and acquiring input information based on user operation. The input interface 33 is, for example, a physical key, a capacitive key, a touchscreen integrally provided with a display of the output interface 34, a microphone for receiving audio input, or the like, but is not limited to these.

The output interface 34 includes one or more output interfaces for outputting information to notify the user. For example, the output interface 34 includes, but is not limited to, a display for outputting information as images, a speaker for outputting information as audio, or the like.

The controller 35 includes at least one processor, at least one dedicated circuit, or a combination thereof. The processor is a general purpose processor such as a CPU or a GPU, or a dedicated processor that is dedicated to specific processing. The dedicated circuit is, for example, an FPGA or an ASIC. The controller 35 executes processes related to operations of the terminal apparatus 30 while controlling the components of the terminal apparatus 30.

The positional information acquisition interface 36 includes at least one receiver compliant with a satellite positioning system. For example, the positional information acquisition interface 36 may include a GPS receiver. The positional information acquisition interface 36 acquires a measured value of the position of the terminal apparatus 30 as positional information. The positional information includes, for example, an address, a latitude, a longitude, an altitude, and the like.

### Operations of Information Processing System

Operations of the information processing system 1 illustrated in FIG. 1 are described with reference to FIGS. 1 to 4.

The patient 5 is a patient in need of treatment in a non-hospital setting, such as at home or on the street. The patient 5 or a third party who notices something wrong with the patient 5 notifies the emergency center 3 that the patient 5 needs treatment.

The information communication device installed in the emergency center 3 transmits the information on the call concerning the patient 5 to the information processing apparatus 10. The information on the call concerning the patient 5 may, for example, include the name of the patient, the date and time the call was received, the position of the patient, the condition of the patient, etc. The “information on the call concerning the patient 5” is also referred to below simply as “call information”.

The communication interface 11 of the information processing apparatus 10 receives the call information transmitted by the information communication device installed in the emergency center 3. The controller 15 acquires the call information, transmitted by the information communication device installed in the emergency center 3, via the communication interface 11.

The memory 12 stores positional information for a plurality of healthcare professionals 2. The healthcare professionals 2 whose positional information is stored by the memory 12 are healthcare professionals who have consented to register their positional information.

The memory 12 stores, in association with the healthcare professional 2, information such as identification information for the terminal apparatus 30 owned by the healthcare professional 2, positional information for the terminal apparatus 30, identification information for the vehicle 20 owned by the healthcare professional 2, and positional information for the vehicle 20.

Here, the positional information for the healthcare professional 2 may be positional information for the terminal apparatus 30 owned by the healthcare professional 2. When the healthcare professional 2 is on board the vehicle 20, the position of the terminal apparatus 30 of the healthcare professional 2 matches the position of the vehicle 20 of the healthcare professional 2. Here, the position of the terminal apparatus 30 of the healthcare professional 2 matching the position of the vehicle 20 of the healthcare professional 2 is not limited to the case of the positions completely matching, but rather includes the case of the position of the terminal apparatus 30 of the healthcare professional 2 being within a predetermined distance from the position of the vehicle 20 of the healthcare professional 2.

The positional information, stored by the memory 12, for the terminal apparatus 30 of the healthcare professional 2 is periodically updated. The positional information for the terminal apparatus 30 of the healthcare professional 2 is periodically updated by the information processing apparatus 10 periodically acquiring the positional information for the terminal apparatus 30. The controller 35 of the terminal apparatus 30 periodically transmits the positional information for the terminal apparatus 30 acquired by the positional information acquisition interface 36 to the information processing apparatus 10 via the communication interface 31.

The positional information for the vehicle 20 of the healthcare professional 2 stored by the memory 12 is periodically updated. The positional information for the vehicle 20 of the healthcare professional 2 is periodically updated by the information processing apparatus 10 periodically acquiring the positional information for the vehicle 20. The control apparatus 22 of the vehicle 20 periodically transmits the positional information for the vehicle 20 acquired by the positional information acquisition apparatus 23 to the information processing apparatus 10 via the communication apparatus 21.

Upon acquiring the call information, the controller 15 generates a message to notify the healthcare professional 2 of the call information. The controller 15 refers to the memory 12 and extracts the information on a healthcare professional 2 who is at a position close to the position of the patient 5. At this time, the controller 15 may extract information on a healthcare professional 2 who is at a position close to the position of the patient 5 based on the positional information for the terminal apparatus 30 of the healthcare professional 2.

The controller 15 transmits the generated message via the communication interface 11 to the healthcare professional 2 who is closest to the position of the patient 5. The controller 15 may transmit the message to the terminal apparatus 30 of the healthcare professional 2 via the communication interface 11. Alternatively, when the position of the terminal apparatus 30 of the healthcare professional 2 who is closest to the position of the patient 5 matches the position of the vehicle 20, and the healthcare professional 2 is therefore inferred to be on board the vehicle 20, the message may be transmitted to the vehicle 20 of the healthcare professional 2 via the communication interface 11. It is assumed below that the controller 15 transmits the message to the terminal apparatus 30 of the healthcare professional 2 via the communication interface 11.

The communication interface 31 of the terminal apparatus 30 receives the message transmitted by the information processing apparatus 10. The controller 35 acquires the message transmitted by the information processing apparatus 10 via the communication interface 31.

The controller 35 controls the output interface 34 to output the message. The message outputted by the output interface 34 includes information on the call concerning the patient 5. The controller 35 may control the output interface 34 to display the message as an image or to output the message as audio.

The input interface 33 accepts input from the healthcare professional 2 regarding the message. The input from the healthcare professional 2 includes information on whether the healthcare professional 2 is able to go to the patient 5.

The controller 35 transmits the information accepted from the healthcare professional 2 by the input interface 33 to the information processing apparatus 10 via the communication interface 31.

The communication interface 11 of the information processing apparatus 10 receives, from the terminal apparatus 30, the input from the healthcare professional 2 regarding the message. The controller 15 acquires the input from the healthcare professional 2 regarding the message via the communication interface 11.

When content acquired from the terminal apparatus 30 includes information indicating an ability to go to the patient 5, the controller 15 transmits first information, which is information notifying that there is a healthcare professional 2 who is going to the patient 5 based on the message, to the emergency center 3.

When the content acquired from the terminal apparatus 30 includes information indicating an inability to go to the patient 5, the controller 15 may transmit a message via the communication interface 11 to the terminal apparatus 30 of the healthcare professional 2 who is at the next closest position to the position of the patient 5. The controller 15 may repeat the process of transmitting a message via the communication interface 11 to the terminal apparatus 30 of the healthcare professional 2 until information indicating an ability to go to the patient 5 is acquired from the terminal apparatus 30. At this time, the controller 15 may transmit messages to the terminal apparatus 30 of the healthcare professional 2 while prioritizing the messages in order of proximity to the position of the patient 5.

Upon acquiring, from the information processing apparatus 10, the first information notifying that there is a healthcare professional 2 who is going to the patient 5, the emergency center 3 can determine the priority of the destination of the ambulance 4 based on the first information.

For example, when a call is received from a patient 5A and a patient 5B, and the first information indicating that there is a healthcare professional 2 who is going to the patient 5A is acquired from the information processing apparatus 10, the destination of the ambulance 4 can be determined so that the ambulance 4 goes to the patient 5B to which the healthcare professional 2 is not going.

For example, when a call is received only from a patient 5A, and the first information indicating that there is a healthcare professional 2 who is going to the patient 5A is acquired from the information processing apparatus 10, the destination of the ambulance 4 can be determined so that the ambulance 4 also goes to the patient 5A to which the healthcare professional 2 is going.

The determination of the destination of the ambulance 4 based on the first information acquired from the information processing apparatus 10 may be made by the staff of the emergency center 3 or may be made automatically, based on an algorithm stored in advance, by the information communication device installed in the emergency center 3.

Upon arriving at the patient 5, the healthcare professional 2 inputs second information, which is information regarding the symptoms of the patient 5, to the input interface 33 of the terminal apparatus 30. The second information, which is information regarding the symptoms of the patient 5, may include information regarding the content of the symptoms, such as what kind of disease or injury the patient has, information regarding the degree of the symptoms, such as whether the patient's symptoms are severe or mild, and the like.

The controller 35 transmits the second information accepted from the healthcare professional 2 by the input interface 33 to the information processing apparatus 10 via the communication interface 31.

The communication interface 11 of the information processing apparatus 10 receives the second information from the terminal apparatus 30.

The controller 15 acquires the second information via the communication interface 11.

The controller 15 transmits the second information acquired from the terminal apparatus 30 to the emergency center 3 via the communication interface 11.

Upon acquiring the second information from the information processing apparatus 10, the emergency center 3 can determine the priority of the destination of the ambulance 4 based on the second information.

For example, when receiving a call from a patient 5A and a patient 5B, if the second information acquired from the healthcare professional 2 who went to the patient 5A includes information that the patient 5A is seriously injured and needs urgent treatment, the emergency center 3 can determine the destination of the ambulance 4 so that the ambulance 4 also goes to the patient 5A.

The determination of the destination of the ambulance 4 based on the second information acquired from the information processing apparatus 10 may be made by the staff of the emergency center 3 or may be made automatically, based on an algorithm stored in advance, by the information communication device installed in the emergency center 3.

The healthcare professional 2 who went to the patient 5 and provided first aid to the patient 5 inputs third information, which is information regarding the condition of the patient 5 after the first aid, to the input interface 33 of the terminal apparatus 30. The third information, which is information regarding the condition of the patient 5 after the first aid, may include information such as whether the patient 5 after the first aid was sent to a hospital, where the patient 5 after the first aid is located if the patient 5 is being made to wait, and the like.

The controller 35 transmits the third information accepted from the healthcare professional 2 by the input interface 33 to the information processing apparatus 10 via the communication interface 31.

The communication interface 11 of the information processing apparatus 10 receives the third information from the terminal apparatus 30. The controller 15 acquires the third information via the communication interface 11.

The controller 15 transmits the third information acquired from the terminal apparatus 30 to the emergency center 3 via the communication interface 11.

Upon acquiring the third information from the information processing apparatus 10, the emergency center 3 can determine the destination of the ambulance 4 based on the third information.

For example, when the third information acquired from the healthcare professional 2 who went to the patient 5A includes information indicating that the patient 5A after the first aid has already left for the hospital, the emergency center 3 can determine the destination of the ambulance 4 so that the ambulance 4 goes to another patient 5, other than the patient 5A.

For example, when the third information acquired from the healthcare professional 2 who went to the patient 5A includes information indicating that the patient 5A after the first aid is waiting on a park bench, the emergency center 3 can determine the destination of the ambulance 4 so that the ambulance 4 goes to the park where the patient 5A is waiting.

The determination of the destination of the ambulance 4 based on the third information acquired from the information processing apparatus 10 may be made by the staff of the emergency center 3 or may be made automatically, based on an algorithm stored in advance, by the information communication device installed in the emergency center 3.

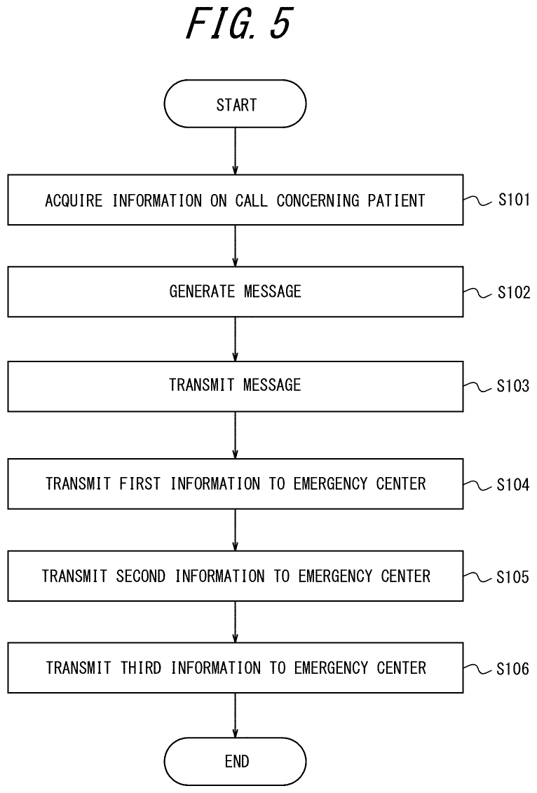

Operations of the information processing system 1 are described with reference to the flowchart in FIG. 5.

In step S101, the controller 15 of the information processing apparatus 10 acquires, via the communication interface 11, information on a call concerning the patient 5, the information being transmitted by the information communication device installed in the emergency center 3.

In step S102, the controller 15 generates a message to notify the healthcare professional 2 of the call information.

In step S103, the controller 15 transmits a message via the communication interface 11 to the healthcare professional 2 who is closest to the position of the patient 5.

In step S104, upon acquiring first information, from the terminal apparatus 30, notifying that there is a healthcare professional 2 who is going to the patient 5 based on the message, the controller 15 transmits the first information to the emergency center 3 via the communication interface 11.

In step S105, upon acquiring second information, which is information regarding the symptoms of the patient 5, from the terminal apparatus 30, the controller 15 transmits the second information to the emergency center 3 via the communication interface 11.

In step S106, upon acquiring third information, from the terminal apparatus 30, which is information regarding the condition of the patient 5 after first aid, the controller 15 transmits the third information to the emergency center 3 via the communication interface 11.

As described above, in the information processing apparatus 10 of the present embodiment, the controller 15 generates a message notifying a healthcare professional 2 of the information on the call concerning the patient 5, and transmits first information, to the emergency center 3, notifying that there is a healthcare professional 2 who is going to the patient 5 based on the message. In this way, the emergency center 3 can learn, from the first information transmitted by the information processing apparatus 10, that there is a healthcare professional 2 who is going to the patient 5. The emergency center 3 can therefore learn which patient 5 the healthcare professional 2 is going to when there is a plurality of patients 5, for example, and can appropriately determine the priority of the destination of the ambulance 4. According to the information processing apparatus 10 of the present embodiment, rescue operations can thus be performed efficiently by collaboration between the ambulance 4 and a healthcare professional 2 other than the healthcare professional on board the ambulance 4.

The present disclosure is not limited to the embodiment described above. For example, a plurality of blocks described in the block diagrams may be integrated, or a block may be divided. Instead of executing a plurality of steps described in the flowcharts in chronological order in accordance with the description, the plurality of steps may be executed in parallel or in a different order according to the processing capability of the apparatus that executes each step, or as required. Other modifications can be made without departing from the spirit of the present disclosure.

For example, some of the processing operations executed in the information processing apparatus 10 in the above embodiment may be executed in the vehicle 20 or the terminal apparatus 30.

For example, some of the processing operations executed in the terminal apparatus 30 in the above embodiment may be executed in the vehicle 20.

For example, in the above embodiment, the information processing apparatus 10 may be installed in the emergency center 3.

For example, a configuration that causes a general purpose electronic device such as a smartphone, a computer, or the like to function as the information processing apparatus 10 according to the embodiment described above is possible. Specifically, a program in which processes for realizing the functions of the information processing apparatus 10 or the like according to the embodiment are written may be stored in a memory of the electronic device, and the program may be read and executed by a processor of the electronic device. Accordingly, in an embodiment, the present disclosure can also be implemented as a program executable by a processor.

In the above embodiment, the case of the healthcare professional 2 boarding the vehicle 20 of the healthcare professional 2 to go to the patient 5 has been described, but the vehicle 20 that the healthcare professional 2 boards is not limited to the vehicle 20 owned by the healthcare professional 2. For example, a third party other than the healthcare professional 2 may let the healthcare professional 2 board a vehicle 20 owned by the third party and may then go to the patient 5. The healthcare professional 2 may also go to the patient 5 on foot, by public transportation, or other means.

In the above embodiment, the healthcare professional 2 is described as going to the patient 5, but the person going to the patient 5 is not limited to the healthcare professional 2. For example, a member of the public who has taken a course in first aid may go to the patient 5.

1. An information processing apparatus comprising a controller configured to:
generate a message notifying a healthcare professional of information on a call concerning a patient; and
transmit first information, to an emergency center, notifying that there is a healthcare professional who is going to the patient based on the message.

2. The information processing apparatus of claim 1, wherein the controller is configured to transmit second information to the emergency center, the second information being information regarding symptoms of the patient and being acquired from the healthcare professional who arrives at the patient.

3. The information processing apparatus of claim 1, wherein the controller is configured to transmit third information to the emergency center, the third information being information regarding a condition of the patient after first aid and being acquired from the healthcare professional who performs first aid on the patient.

4. The information processing apparatus of claim 1, wherein when a position of a terminal apparatus of the healthcare professional matches a position of a vehicle of the healthcare professional, the controller is configured to judge that the healthcare professional is on board the vehicle.

5. The information processing apparatus of claim 4, wherein the controller is configured to transmit the message to the terminal apparatus or the vehicle.

6. The information processing apparatus of claim 1, further comprising a memory configured to store positional information for a plurality of healthcare professionals.

7. The information processing apparatus of claim 6, wherein the controller is configured to transmit the message to the healthcare professional whose positional information is close to a position of the patient.

8. A non-transitory computer readable medium storing a program configured to cause a computer to execute operations comprising:
generating a message notifying a healthcare professional of information on a call concerning a patient; and
transmitting first information, to an emergency center, notifying that there is a healthcare professional who is going to the patient based on the message.

9. The non-transitory computer readable medium of claim 8, wherein the operations further comprise transmitting second information to the emergency center, the second information being information regarding symptoms of the patient and being acquired from the healthcare professional who arrives at the patient.

10. The non-transitory computer readable medium of claim 8, wherein the operations further comprise transmitting third information to the emergency center, the third information being information regarding a condition of the patient after first aid and being acquired from the healthcare professional who performs first aid on the patient.

11. The non-transitory computer readable medium of claim 8, wherein the operations further comprise judging, when a position of a terminal apparatus of the healthcare professional matches a position of a vehicle of the healthcare professional, that the healthcare professional is on board the vehicle.

12. The non-transitory computer readable medium of claim 11, wherein the operations further comprise transmitting the message to the terminal apparatus or the vehicle.

13. The non-transitory computer readable medium of claim 8, wherein the operations further comprise storing positional information for a plurality of healthcare professionals.

14. The non-transitory computer readable medium of claim 13, wherein the operations further comprise transmitting the message to the healthcare professional whose positional information is close to a position of the patient.

15. An information processing method comprising:
generating a message notifying a healthcare professional of information on a call concerning a patient; and
transmitting first information, to an emergency center, notifying that there is a healthcare professional who is going to the patient based on the message.

16. The information processing method of claim 15, further comprising transmitting second information to the emergency center, the second information being information regarding symptoms of the patient and being acquired from the healthcare professional who arrives at the patient.

17. The information processing method of claim 15, further comprising transmitting third information to the emergency center, the third information being information regarding a condition of the patient after first aid and being acquired from the healthcare professional who performs first aid on the patient.

18. The information processing method of claim 15, further comprising judging, when a position of a terminal apparatus of the healthcare professional matches a position of a vehicle of the healthcare professional, that the healthcare professional is on board the vehicle.

19. The information processing method of claim 18, further comprising transmitting the message to the terminal apparatus or the vehicle.

20. The information processing method of claim 15, further comprising storing positional information for a plurality of healthcare professionals.
